(19) United States
(12) Patent Application Publication
(10) Pub. No.: US 2012/0330225 A1
(43) Pub. Date: Dec. 27, 2012

(54) Active Catheter

(75) Inventor: Sean Farrell, Fresno, CA (US)

(73) Assignee: Fresenius Medical Care Holdings, Inc., Waltham, MA (US)

(21) Appl. No.: 13/169,551

(22) Filed: Jun. 27, 2011

(51) Int. Cl. A61M 1/28

(52) U.S. Cl. 604/29

(57) ABSTRACT

A catheter assembly includes a tubular member having a first connector at its distal end for coupling with the proximal end of a catheter. The tubular member has at least one external guide wire that is slidably contained inside the tubular member. The at least one external guide wire has an attachment member for attaching to an internal guide wire catch that is slidably contained inside the catheter, whereby manipulation of the guide wire causes motion in the distal end of the catheter. The catheter assembly can be articulated within an internal cavity of a patient to more completely and accurately fill and/or drain the cavity during, for example, peritoneal dialysis.

### BACKGROUND OF THE INVENTION

Renal dysfunction or failure and, in particular, end-stage renal disease, causes the body to lose the ability to remove water and minerals and excrete harmful metabolites, maintain acid-base balance, and control electrolyte and mineral concentrations within physiological ranges. Toxic uremic waste metabolites including urea, creatinine, uric acid, and phosphorus accumulate in the body's tissues, which can result in a person's death if the filtration function of the kidney is not replaced.

Dialysis is commonly used to replace kidney function by removing these waste toxins and excess water. In one type of dialysis treatment, hemodialysis, toxins are removed from a patient's blood externally in a hemodialysis machine. Blood passes from the patient through a dialyzer separated by a semi-permeable membrane from an externally-supplied dialysate. Waste and toxins dialyze out of the blood through the semi-permeable membrane into the dialysate, which is then discarded. Hemodialysis treatment typically lasts several hours and must be performed under medical supervision three or four times a week, requirements that significantly decrease a patient's autonomy and quality of life. Also, since hemodialysis is performed periodically instead of continuously, the patient's condition and general well-being tend to be poor both immediately before hemodialysis (when toxin levels are high) and after hemodialysis (when electrolytes are imbalanced), resulting in the patient having symptoms that range from nausea and vomiting to edema.

Peritoneal dialysis is another type of dialysis treatment used to replace kidney function in which sterile, pyrogen-free dialysis solution is infused into the patient's peritoneal cavity. The peritoneal membrane serves as a natural dialyzer and toxic uremic waste metabolites and various ions diffuse from the patient's bloodstream across the membrane into the dialysis solution due to their concentration gradients. At the same time, water is drawn into the peritoneal cavity by an osmotic gradient. The dialysis solution is typically removed, discarded and replaced with fresh dialysis solution on a semi-continuous or continuous basis. Inflow to and outflow from the patient's peritoneal cavity of the dialysis solution is typically accomplished by one or more access ports, where the access ports can include medically appropriate plastic tubing, a double lumen catheter or two single lumen catheters.

The traditional catheter is a relatively simple tube which is surgically implanted into the patient's abdominal cavity. This tube is typically stationary, and only enables filling and draining of liquid (e.g., dialysis solution) in its direct vicinity. It is possible for a patient to have trapped liquid pockets that cannot be drained effectively with a stationary catheter. Additionally, some patients develop fibrous tissue that clogs the small inlet holes at the tip of the catheter. Clogging of these holes presents more drain and fill complications because liquid is thereby hindered from entering or exiting the patient's abdominal cavity.

Therefore, there is a need for a catheter that reduces or eliminates the above mentioned problems.

### SUMMARY OF THE INVENTION

The invention generally is directed to a catheter that can be articulated within an internal cavity of a patient to more completely and accurately fill and/or drain the cavity. Examples of internal cavities of a patient include the bladder and the peritoneal cavity.

In one embodiment, a catheter assembly includes a tubular member having a first connector at its distal end for coupling with the proximal end of a catheter. The tubular member has at least one external guide wire that is slidably contained inside the tubular member. The at least one external guide wire has an attachment member for attaching to an internal guide wire catch that is slidably contained inside the catheter, whereby manipulation of the guide wire causes motion in the distal end of the catheter. The first connector can be a male connector, and the proximal end of the catheter can include a female connector. The catheter assembly can include a catheter for flowing liquid into and out of an internal cavity of a patient, the catheter housing a liquid-flow tube, at least one internal guide wire that is slidably contained inside the liquid-flow tube, and at least one guide-wire catch that is slidably contained inside the catheter. In one embodiment, the liquid is dialysis solution, and the internal cavity is the peritoneal cavity of the patient. The at least one guide-wire catch can include a tab configured to connect to the internal guide wire. In some embodiments, the attachment member on the external guide wire can be angled to slidably connect to a step on the guide wire catch.

The catheter assembly further includes at least one wire guide located within, positioned along, and connected to an inside surface of the liquid-flow tube, wherein the at least one internal guide wire is slidably contained within the at least one wire guide, and is connected to the liquid-flow tube at a distal end of the wire guide. In some embodiments, the wire guide can be a tube, the tube being closed at the distal end. In other embodiments, the wire guide can include a series of tabs with guide openings.

In certain embodiments, the at least one wire guide can include two wire guides positioned at 90 degrees from each other with respect to the center of the liquid-flow tube, each wire guide including one internal guide wire that is slidably contained within the wire guide, and connected to the liquid-flow tube at the distal end of the wire guide.

In another embodiment, a catheter system includes a tubular member as described above, a catheter as described above, and a guide wire actuator. The guide wire actuator can include an apparatus for pumping dialysis solution between a peritoneal dialysis machine and a patient, in order to perform peritoneal dialysis on the patient.

In yet another embodiment, a method of articulating a catheter includes inserting a tubular member having a first connector at its distal end and at least one external guide wire that is slidably contained inside the tubular member into a proximal end of a catheter, the catheter housing a liquid-flow tube, at least one internal guide wire that is slidably contained inside the liquid-flow tube, and at least one guide-wire catch that is slidably contained inside the catheter, thereby forming a catheter assembly. The method further includes extending the at least one external guide wire until the at least one guide-wire catch engages the external guide wire, thereby forming a guide-wire assembly, and moving the guide-wire assembly longitudinally to articulate the catheter.

The invention has many advantages, including the ability to drain trapped liquid pockets within an internal cavity of a patient, and the prevention of growth and/or detachment of fibrous tissue from around the catheter by articulation of the catheter.

### BRIEF DESCRIPTION OF THE DRAWINGS

The foregoing will be apparent from the following more particular description of example embodiments of the invention, as illustrated in the accompanying drawings in which like reference characters refer to the same parts throughout the different views. The drawings are not necessarily to scale, emphasis instead being placed upon illustrating embodiments of the present invention.

FIG. 1A is an illustration of a tubular member according to this invention.

FIG. 1B is an illustration of a tubular member (i.e., disposable connector) including two external guide wires according to this invention.

FIG. 2 is an illustration of a catheter assembly according to this invention.

FIG. 3 is an illustration of a guide wire catch according to this invention.

FIG. 4 is an illustration of the catheter assembly according to this invention with the barb contacting the guide wire catch.

FIG. 5 is a cross-section of a catheter assembly according to this invention.

FIG. 6 is an illustration of a liquid flow tube and a wire guide tube according to this invention.

FIG. 7 is an illustration of a liquid flow tube and a wire guide including tabs with guide openings according to this invention.

FIG. 8 is a cross-section of a catheter assembly according to this invention with two wire guides.

FIG. 9 is an illustration of a liquid flow tube and two wire guide tubes according to this invention.

FIG. 10 is an illustration of a catheter system including a guide wire actuator assembly according to this invention.

FIG. 11 is an illustration of a Liberty® cycler.

FIG. 12 is an illustration of sideways movement of the catheter according to this invention.

FIG. 13 is an illustration of sideways and upwards movement of the catheter according to this invention.

FIG. 14 is a plan view of a guide wire catch according to this invention.

FIG. 15 is a side view of a guide wire catch according to this invention.

FIG. 16 is an illustration of a connected catheter assembly according to this invention.

FIG. 17 is an illustration of a connected catheter assembly according to this invention with the barb contacting the guide wire catch.

FIG. 18 is an illustration of a locked guide wire catch according to this invention with the barb contacting the guide wire catch.

FIG. 19 is an illustration of a guide wire catch connected to the disposable guide wire according to this invention.

FIG. 20 is an illustration of a guide wire catch moved all the way forward according to this invention.

FIG. 21 is an illustration of a guide wire catch moved all the way backward according to this invention.

FIG. 22 is an illustration of a catheter assembly with two guide wires according to this invention.

FIG. 23 is an illustration of the catheter assembly with two guide wires according to this invention.

FIG. 24 is a plan view of the guide wire catches for two guide wires according to this invention.

FIG. 25 is a plan view of a guide wire catch for one of the two guide wires according to this invention.

FIG. 26 is a plan view of a catheter assembly and one guide wire catch for the two guide wire version according to this invention.

FIG. 27 is a cross-section of a catheter assembly for the two guide wire version according to this invention.

FIG. 28 is side view of a catheter assembly with one guide wire all the way in and the other guide wire all the way out according to this invention.

FIG. 29 is side view of a catheter assembly with the guide wire catch locked in place according to this invention.

FIG. 30 is a plan view of the active catheter cassette according to this invention.

FIG. 31 is a plan view of the active catheter cassette with the guide wire retracted according to this invention.

FIG. 32 is a plan view of the active catheter cassette with the guide wire extended according to this invention.

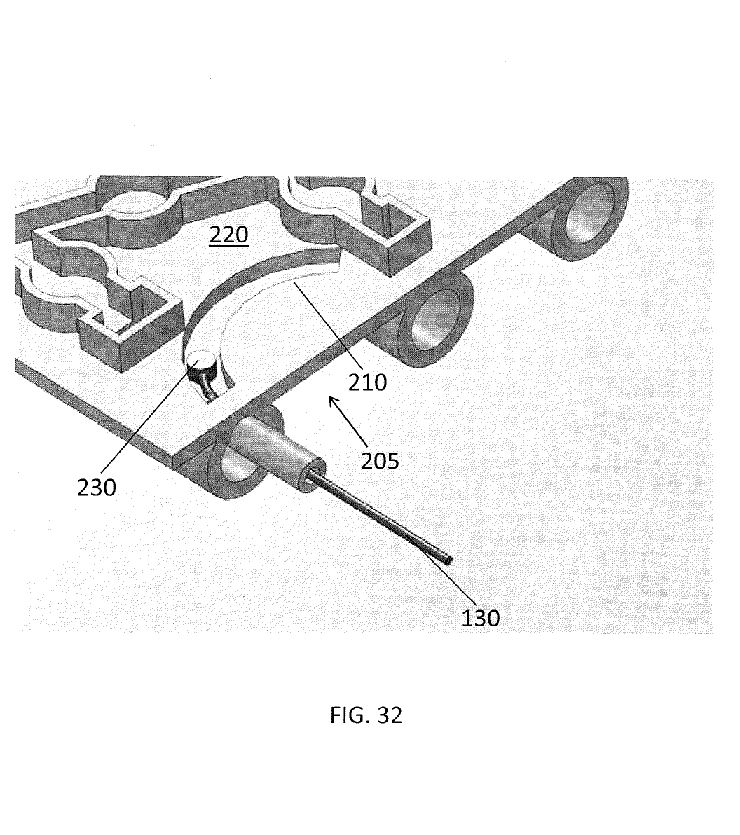

FIG. 33 is an illustration of a catheter system including a guide wire actuator assembly that includes two guide wires according to this invention.

FIG. 34 is a plan view of the active catheter cassette including two guide wires according to this invention.

FIG. 35 is a plan view of the active catheter cassette with independently positioned guide wires according to this invention.

FIG. 36 is an illustration of a flow chart of a method of articulating a catheter according to this invention.

### DETAILED DESCRIPTION OF THE INVENTION

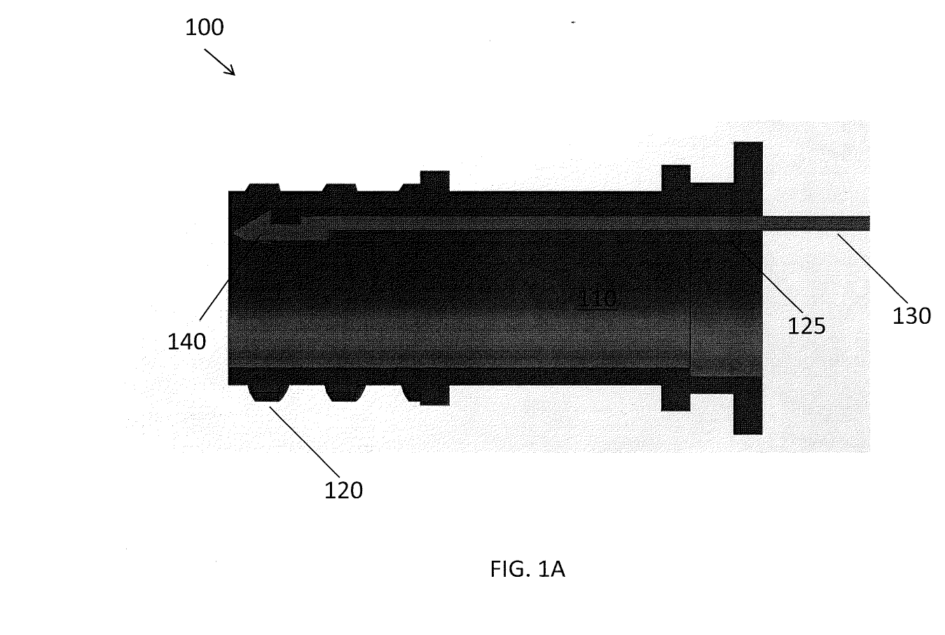

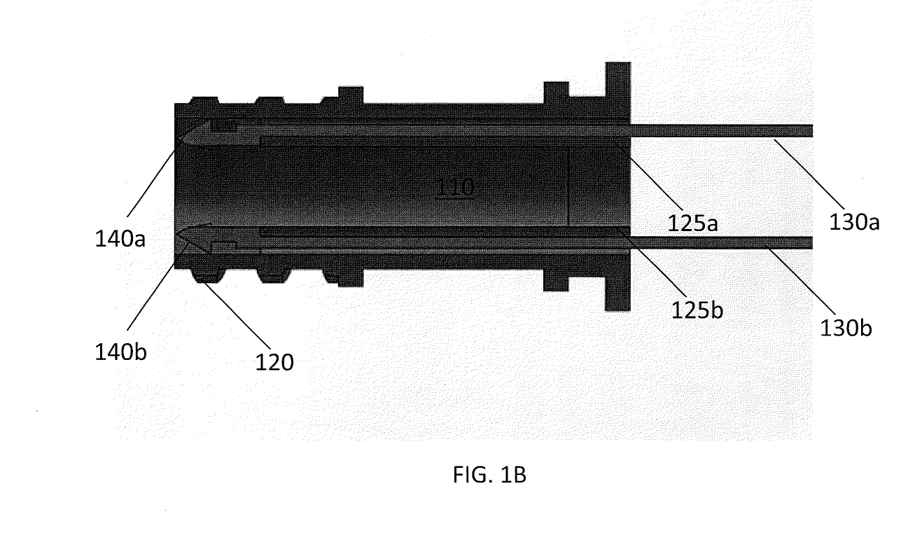

In one embodiment, shown in FIG. 1A, catheter assembly 100 includes tubular member (i.e., disposable connector) 110 having a first connector 120 at its distal end for coupling with the proximal end of a catheter (not shown). Tubular member 110 includes an external guide wire 130 that is slidably contained inside tubular member 110 by a channel 125. The external guide wire 130 has attachment member 140 for attaching to an internal guide wire catch (not shown) that is slidably contained inside the catheter, whereby manipulation of guide wire 130 causes motion in the distal end of the catheter. Manipulation of guide wire 130 can be manual or, alternatively, by a guide wire actuator such as that described below.

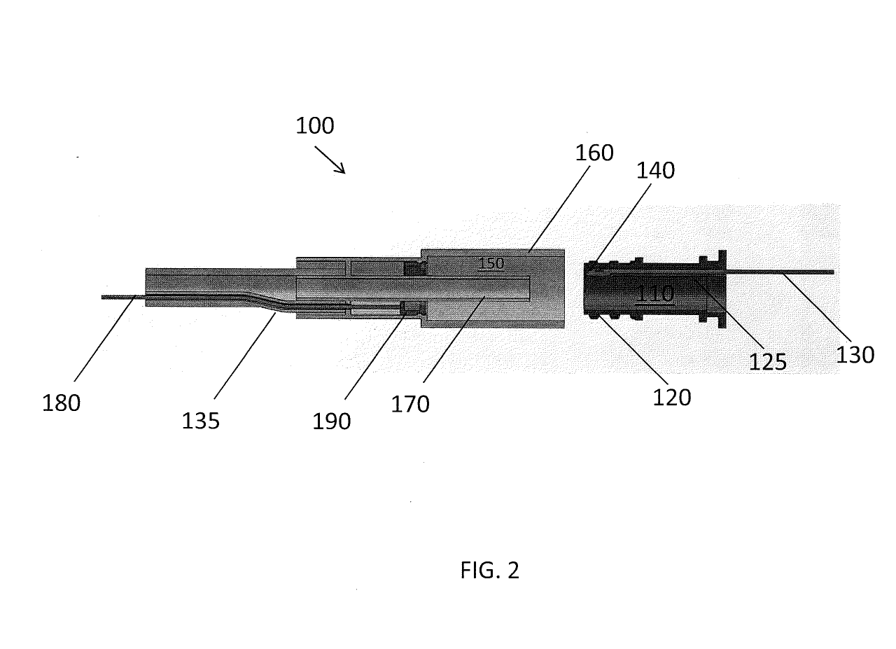

As shown in FIG. 1A, first connector 120 can be a male connector, and, as shown in FIG. 2, the proximal end of patient catheter 150 can include female connector (i.e., patient connector) 160. Alternatively, the first connector can be a female connector, and the proximal end of patient catheter 150 can include a male connector.

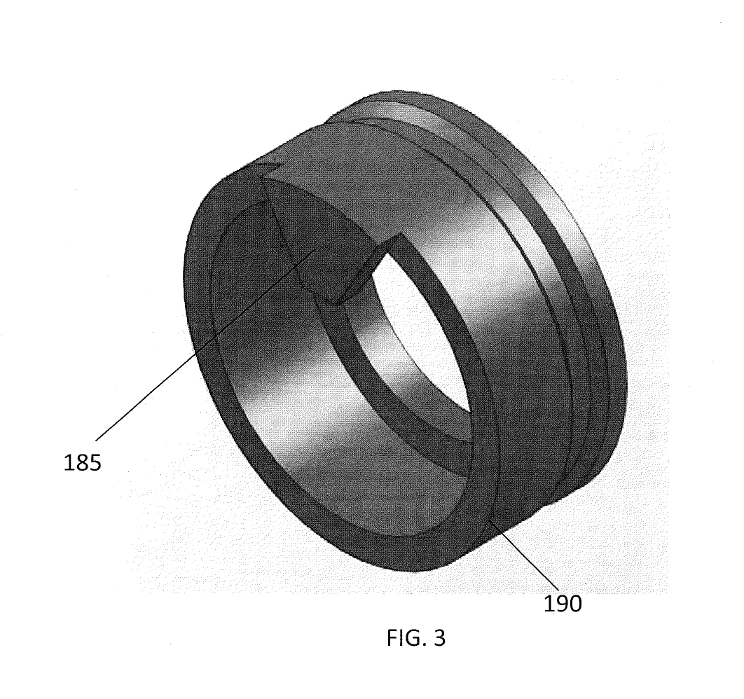

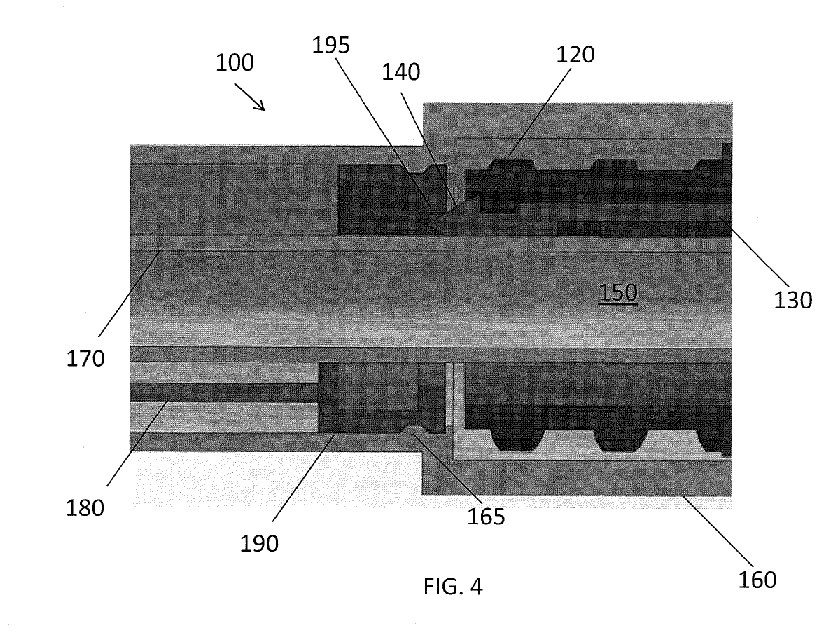

Turning to FIG. 2, catheter assembly 100 can include a patient catheter 150 for flowing liquid into and out of an internal cavity of a patient, catheter 150 housing liquid-flow tube 170, an internal guide wire 180 that is slidably contained inside liquid-flow tube 170, and a guide-wire catch 190 that is slidably contained inside catheter 150. As shown in FIG. 3, guide-wire catch 190 can include tab 185 configured to connect to internal guide wire 180. As shown in greater detail in FIG. 4, in some embodiments, attachment member 140 on external guide wire 130 can be angled to slidably connect to step 195 on guide wire catch 190. Alternatively, the attachment member on the external guide wire can be a step, and the guide wire catch can be angled. In one embodiment, the liquid is dialysis solution, and the internal cavity is the peritoneal cavity of the patient. A variety of peritoneal dialysis solutions can be used (e.g., Delflex®), these solutions being commercially available (e.g., Fresenius Medical Care North America, Waltham Mass.) and well-known in the art.

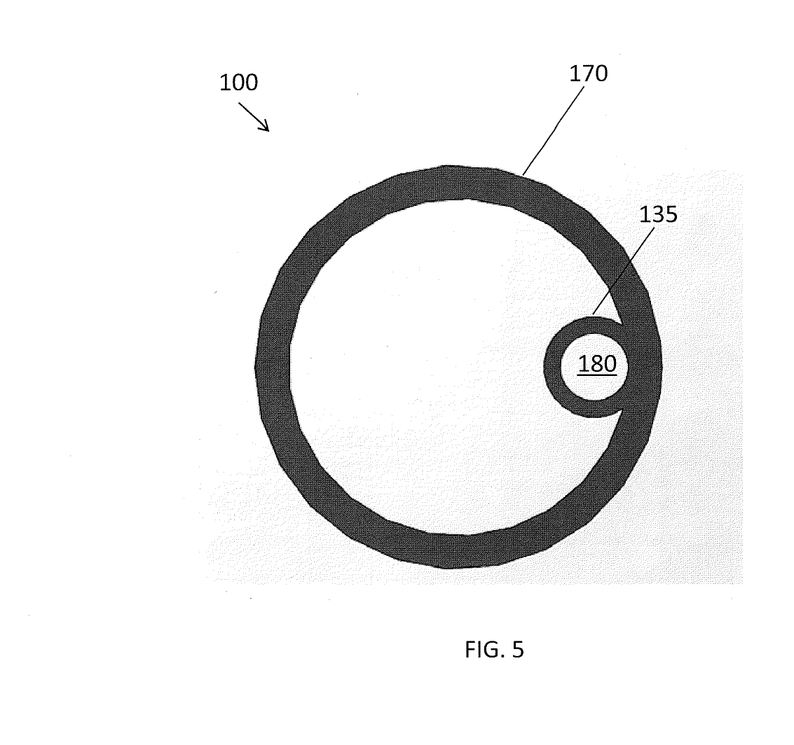

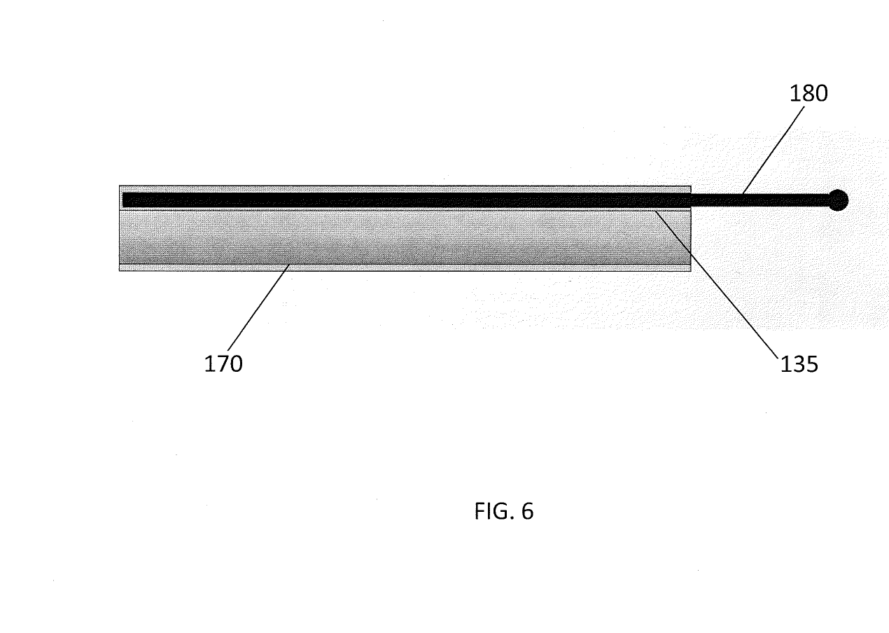

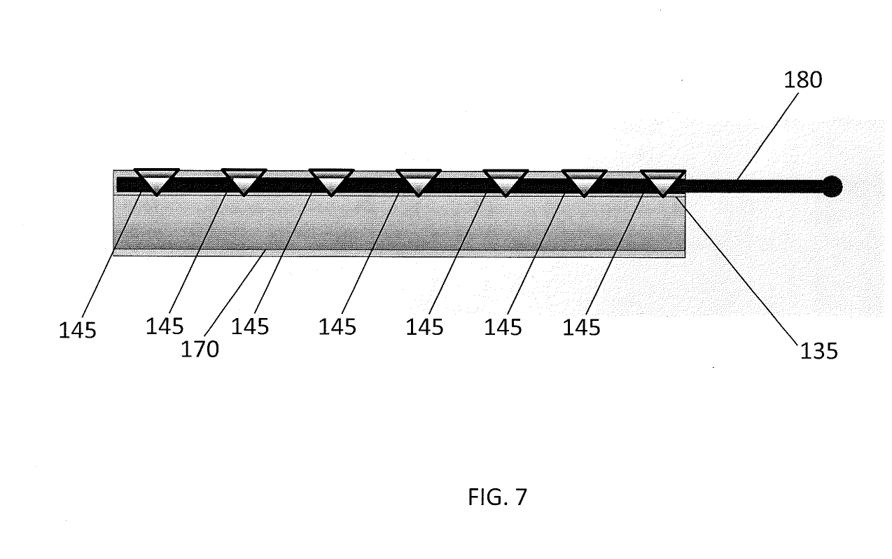

Turning to the cross-section shown in FIG. 5, catheter assembly 100 further includes wire guide 135 located within, positioned along, and connected to an inside surface of liquid-flow tube 170, wherein the internal guide wire 180 is slidably contained within the wire guide 135, and is connected to liquid-flow tube 170 at a distal end of wire guide 135. As shown in FIG. 6, in some embodiments, wire guide 135 can be a tube, the tube being closed at the distal end. In other embodiments, as shown in FIG. 7, wire guide 135 can include a series of tabs 145 with guide openings.

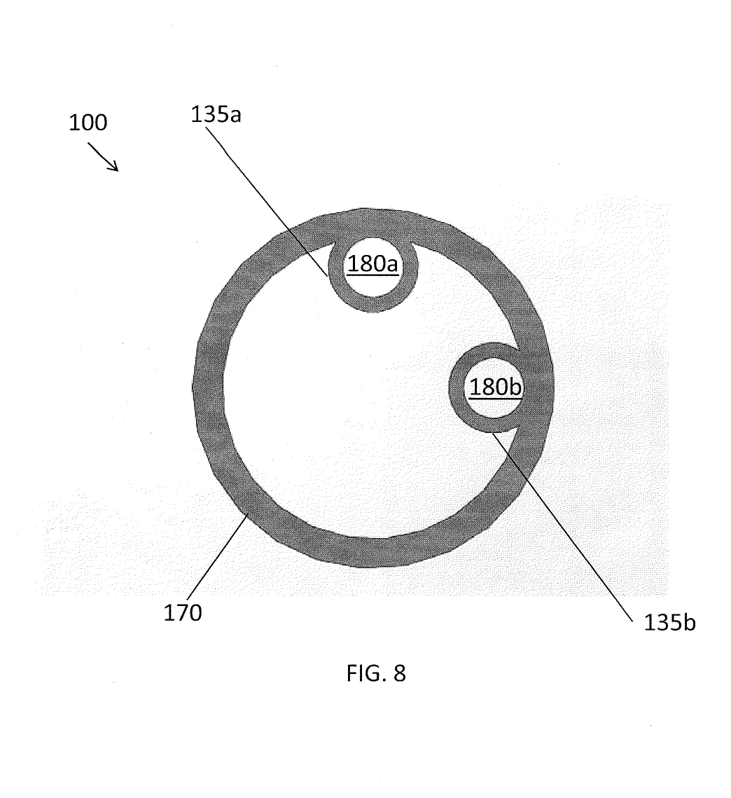

As shown in cross-section in FIG. 8, in certain embodiments, the liquid flow tube 170 can include two wire guides 135a and 135b positioned at 90 degrees from each other with respect to the center of liquid-flow tube 170, each housing one internal guide wire 180a or 180b that is slidably contained within wire guide 135a or 135b, and connected to the liquid-flow tube 170 at the distal end of wire guide 135a or 135b. As shown in FIG. 1B, in the two guide wire configuration, tubular member 110 includes two channels 125a and 125b within it for the two external guide wires 130a and 130b to ride within. The two channels 125a and 125b are positioned at about 180 degrees from each other with respect to the center of tubular member 110, as further described below.

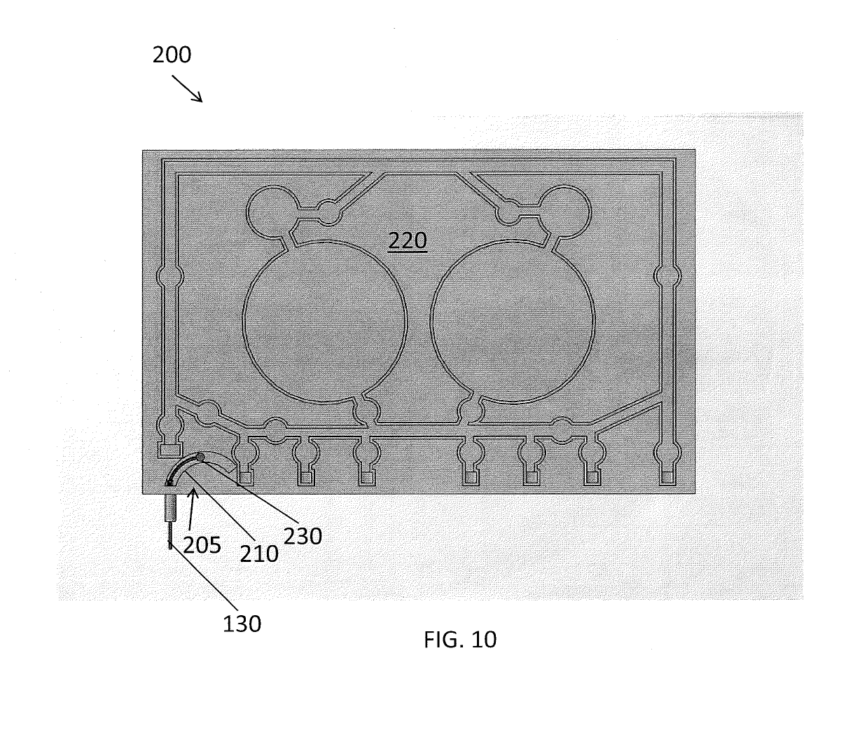

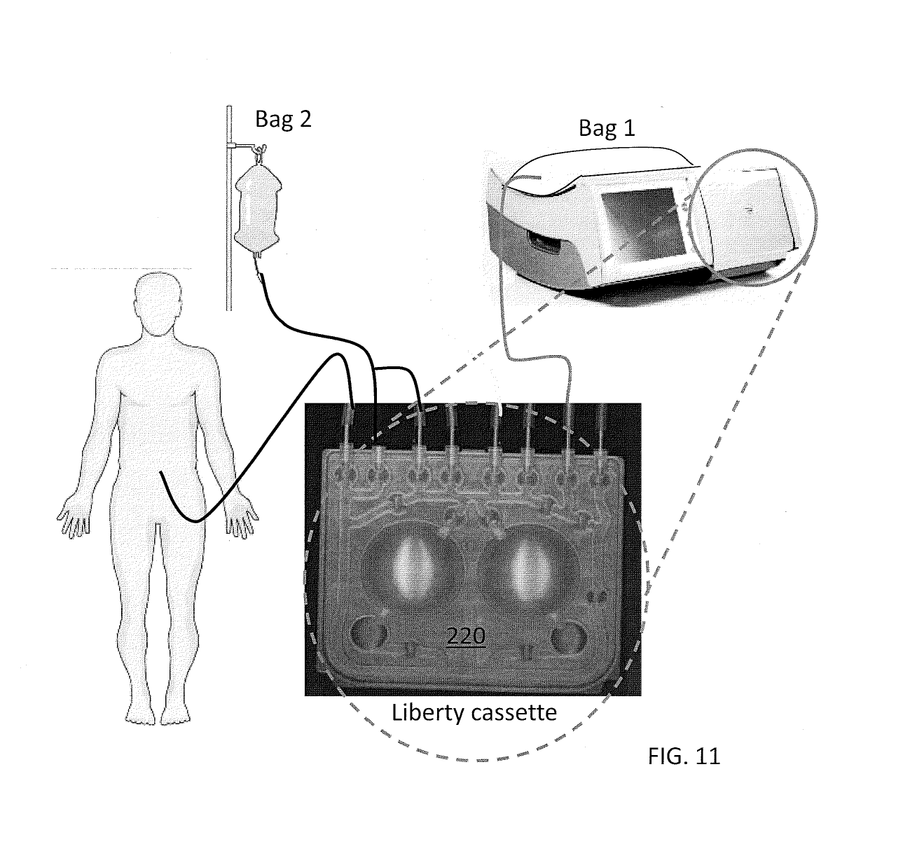

In another embodiment, shown in FIG. 10, catheter system 200 includes a tubular member as described above (not shown), a catheter as described above (not shown), and a guide wire actuator assembly 205 for moving external guide wire 130. Guide wire actuator assembly 205 can include apparatus 220 for pumping dialysis solution between a peritoneal dialysis machine and a patient, in order to perform peritoneal dialysis on a patient. A suitable apparatus is described in U.S. application Ser. Nos. 11/513,618 and 11/515,359, both filed Aug. 31, 2006, and available as the Liberty® cycler from Fresenius Medical Care North America (Waltham, Mass.). Briefly, the cycler transfers predetermined amounts of peritoneal dialysis solution in and out of the patient's peritoneal cavity, and in and out of several bags attached to the cycler, by means of cassette 220, shown in FIG. 11, which controls the flow paths for the respective transfers of dialysis solution. As shown in FIG. 11, bag 1 containing dialysis solution is located on top of the cycler. The top of the cycler also includes a scale, which enables checking for the delivery of predetermined weights (and hence volumes) of dialysis solution by comparing the weight against volume calculations based on the linear travel of the linear actuators (i.e., pumps) of the Liberty® cycler. The scale also includes a heater, which enables the delivery of warm dialysis solution into the patient's peritoneal cavity.

Turning back to FIG. 2, the components of the catheter assembly 100 include tubular member 110, catheter 150, guide wires 180 and 130, and external drive mechanism (described below). The construction of the catheter assembly 100 in the single wire configuration is relatively simple. The catheter can be a tube extrusion, typically in a range of between about 35 cm and about 75 cm in length, with a channel 135 for guide wire 180 and main channel (i.e., liquid-flow tube) 170 for liquid. The internal guide wire 180 is attached to the catheter 150 at the tip, with the rest of the wire 180 free to move forward and back within the wire guide, as illustrated in FIG. 6.

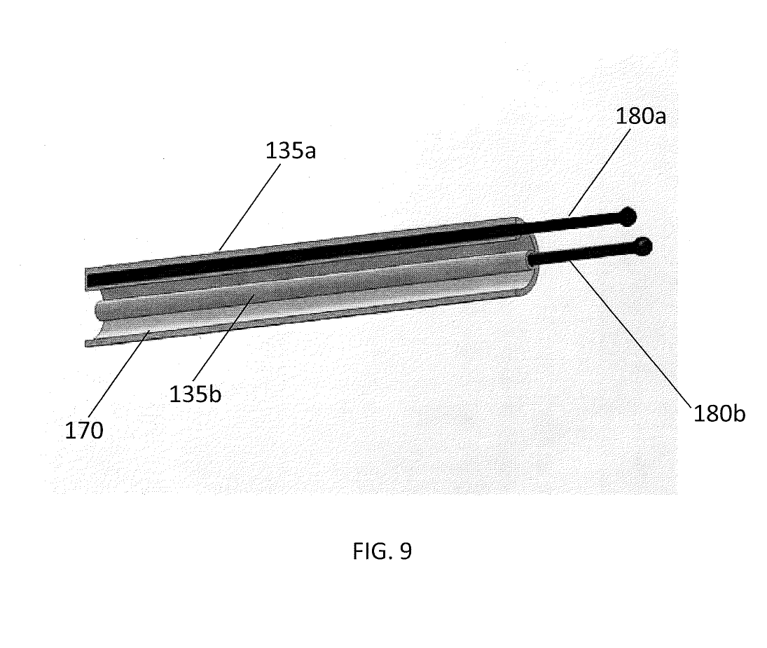

Construction of the double wire configuration is relatively simple as well. The catheter 150 can be a tube extrusion with two channels 135a and 135b for the guide wires 180a and 180b and a main channel (i.e., liquid-flow tube) 170 for liquid. As illustrated in FIG. 9, guide wires 180a and 180b are attached to the catheter at the tip, with the rest of the wires 180a and 180b free to move forward and back within the wire guides 135a and 135b.

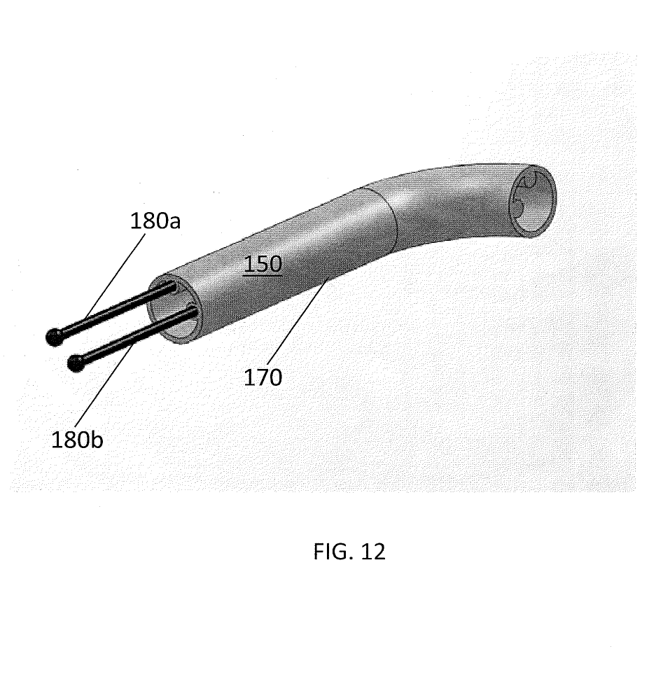

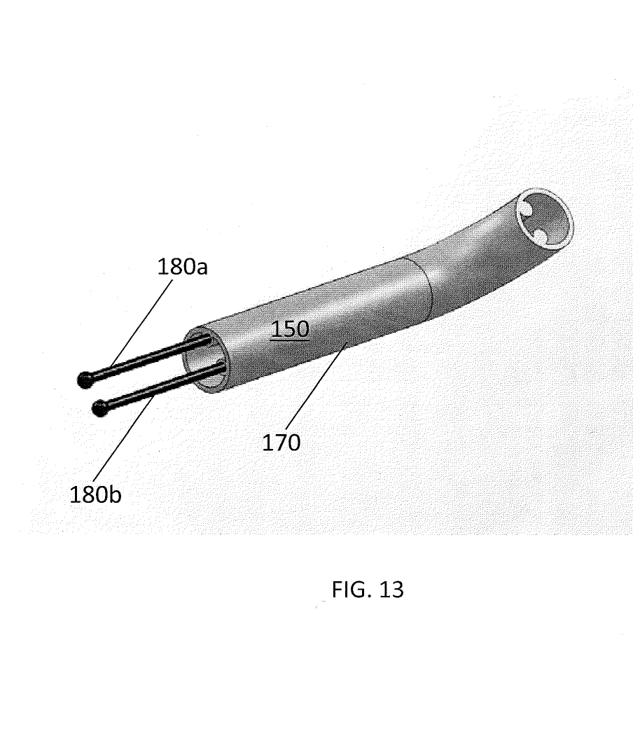

Movement of the active catheter is relatively simple The end of the guide wires 180 or 180a and 180b (shown with a ball end in FIGS. 6, 7, and 9 for illustration purposes) are typically attached to a drive mechanism (such as a linear actuator, rotary motion electric motor, or the like) through the external guide wires 130 or 130a and 130b, which generate the longitudinal pushing and pulling motion. As illustrated in FIG. 12, when the guide wire 180b is pulled, the catheter 150 bends toward the guide wire 180b on the inside of the bend. When the wire 180b is pushed, the catheter 150 bends away from the guide wire 180b on outside of the bend (not shown). If wires 180a and 180b are pulled for three-dimensional movement, the motion of catheter 150 is the same as the motion shown in FIG. 12, but with vertical movement of catheter 150 in addition to horizontal movement, as shown in FIG. 13. Navigation of the catheter can be accomplished by passive computer algorithm, or, alternatively, by active imaging using ultrasound or other similar imaging technology.

In order to prevent patient discomfort from a moving catheter, the motion of the catheter can be reduced to two potential modes. The first mode would be to move the catheter slowly enough such that the motion is nearly imperceptible to the patient. The second mode would be to move the catheter only during the last pumping strokes of a drain or fill, when the patient can experience mild perceptions of liquid movement. During the second mode of movement, the motion of the catheter would still be only slightly perceptible or imperceptible by the patient due to the liquid movement sensation already present.

The basic components of the catheter assembly 100 for the single wire version are as follows (with examples of suitable respective materials):

1. disposable connector 110—plastic (e.g., polypropylene)

2. disposable guide wire 130—stainless steel or plastic filament

3. disposable guide wire barb 140—stainless steel or suitable plastic

4. patient connector 160—plastic (e.g., polypropylene)

5. guide wire catch 190—plastic (e.g., polypropylene or other suitable material), or stainless steel

6. patient guide wire 180—stainless steel or plastic filament

7. patient catheter 150—plastic (e.g., polypropylene)

The attachment method between the patient and the disposable connector is relatively simple To the patient, there will be no extra operation required beyond the simple screw connection they already make. All of the connection components are housed within the patient (internal) connector/catheter and the disposable (external) connector/disposable line.

FIG. 2 shows the assembly as it would exist before connection of the patient connector 160 to the disposable connector 110. The patient, during connection of the disposable connector 160 to the patient connector 110, would connect as they currently do with the simple screw connector threads 120. No extra action is required.

The disposable connector 110 has a similar construction to the existing connector currently in use with the Liberty® cycler cassette 220, with the addition of a similar feature to the catheter tube 150 referred to previously, that is, an internal channel 125 for the guide wire 130 to ride within. A close up illustration of the disposable connector 110 is shown in FIGS. 1A and 1B.

As shown in FIG. 1A, the disposable connector 110 includes channel 125 within it for the guide wire 130 to ride within. The channel 125 stops short of the end of the connector 110, allowing room for the barb 140 on the end of the disposable (i.e., external) guide wire 130 (barb 140 used for connecting the disposable guide wire 130 to the guide wire catch 190 shown in FIG. 2). When disconnected, the disposable guide wire 130 and disposable barb 140 are fully within the disposable connector 110, preventing injury to the patient from the barb 140, and preventing damage to the barb 140.

As shown in FIG. 2, the patient catheter 150 houses the active parts enabling the connection between the disposable guide wire 130 and the patient guide wire 180. The patient catheter 150 houses the guide wire catch 190, which connects the patient guide wire 180 to the disposable guide wire 130. The patient catheter 150 has a channel 135 within it for the patient guide wire 180 to ride within. The patient guide wire 180 is connected to the guide wire catch 190, passes through a small hole in the wall of the patient catheter 150, separating the connection area from the tubing connection area, and into the rest of patient catheter 150. Any motion of the guide wire catch 190 is transmitted to the patient guide wire 180.

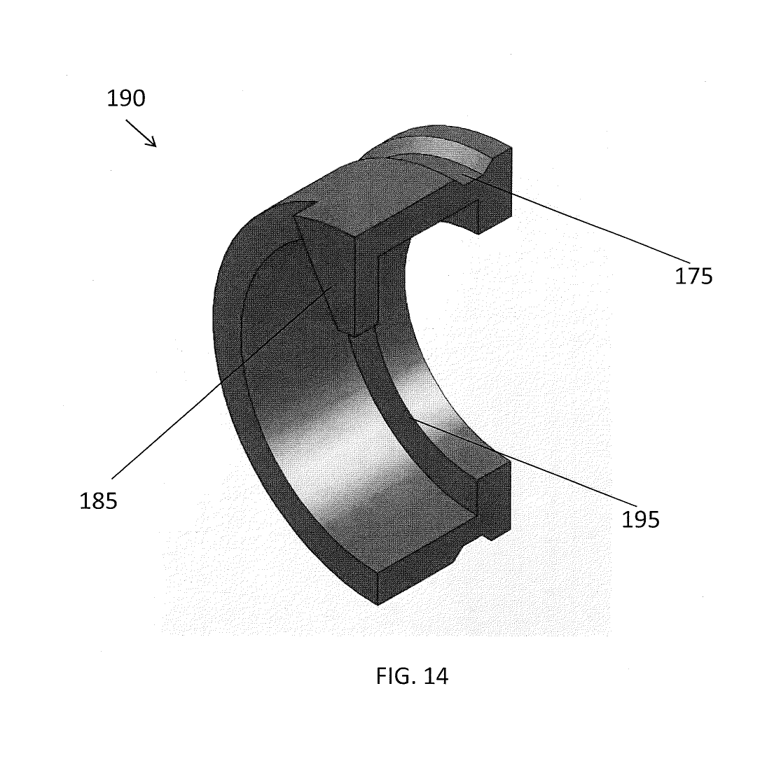

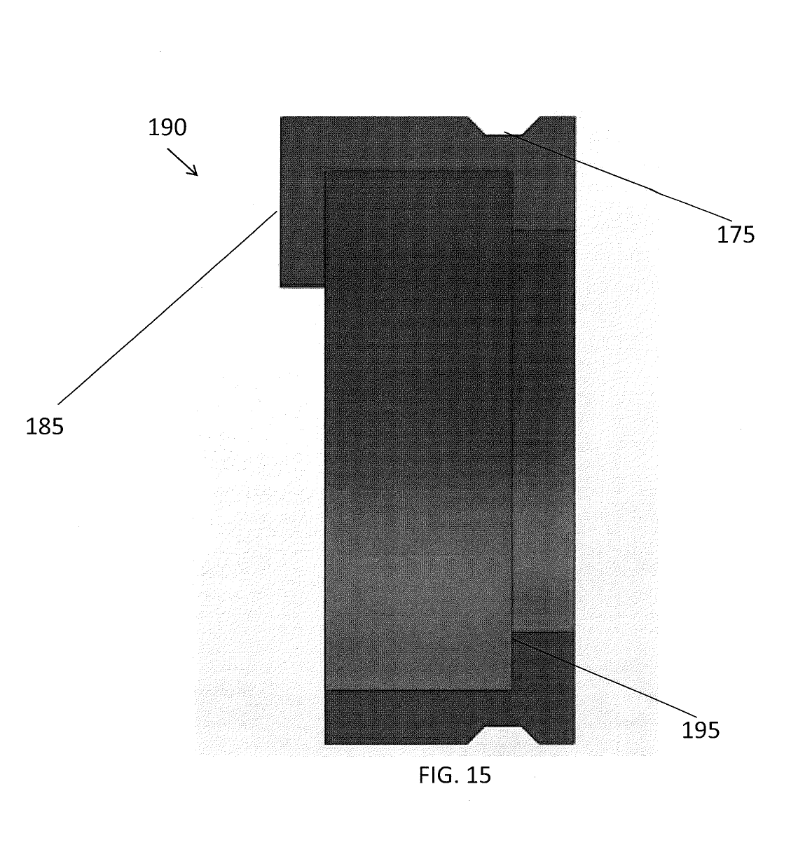

The purpose of the guide wire catch 190 is to transmit the pushing and pulling forces from the disposable guide wire (i. e., external guide wire) 130 to the patient guide wire (i.e., internal guide wire) 180. It also has to allow for the disposable guide wire 130 to connect and disconnect easily, without patient interaction, preferably without altering the catheter/disposable connection method currently used by patients. The current method of catheter/disposable connection is by way of a simple screw fitting. As a result, the guide wire catch 190 must enable the disposable guide wire 130 to connect in any position in a 360 degree circle around the axis of the connectors (alignment is not guaranteed since the patient is screwing one fitting into another, and the fittings are double started). One result of these restrictions is the guide wire catch 190 shown close up in FIGS. 14 and 15. As shown in FIGS. 14 and 15, guide wire catch 190 has a small tab 185 on the back to allow connection to the patient guide wire 180. The small lip 195 seen in FIGS. 14 and 15 is a catch to hold the disposable guide wire barb 140, and allow for the forward and backward movement of the guide wire catch 190, and as a result, the patient guide wire 180. The guide wire catch 190 also has a groove 175 cut circumferentially around the perimeter (i.e., guide wire catch lock groove), providing a seating surface for the guide wire catch lock 165, shown in FIG. 4. The guide wire catch lock 165 allows the disposable guide wire 130 to engage the guide wire catch 190 without moving the catheter. The guide wire catch lock 165 will be described further below.

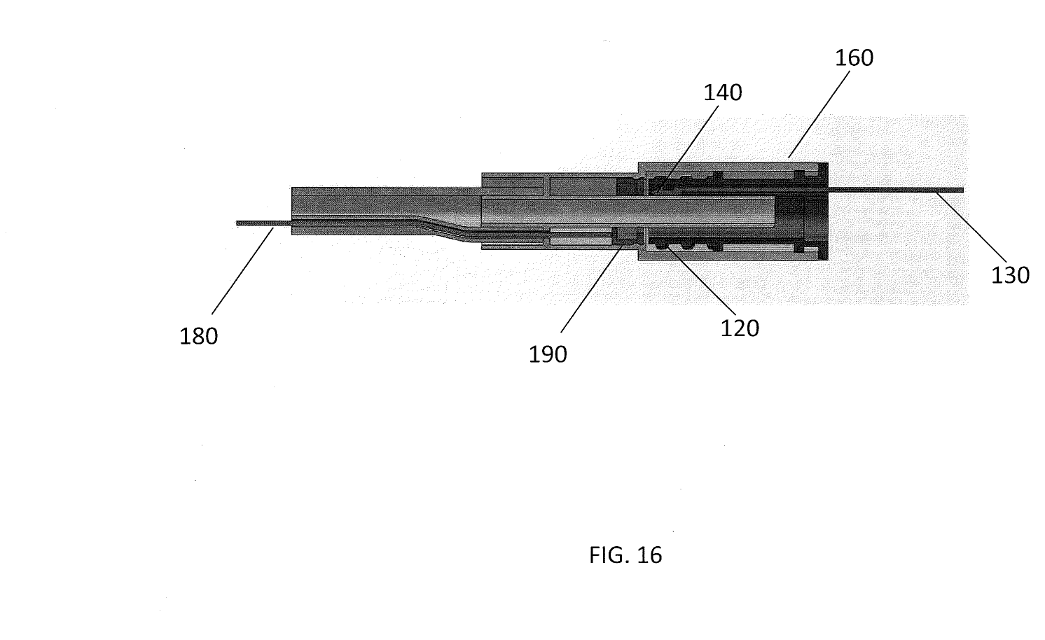

Connection of the disposable connector 110 and the patient connector 160 is made as follows: the patient takes the screw fittings and screws the disposable connector threads 120 into the patient connector 160. The connected assembly is shown in FIG. 16. After the patient has connected the disposable connector 110 and the patient connector 160, the disposable guide wire 130 and guide wire catch 190 are still not connected. The connection takes place by means of an automated connection process facilitated by the ^{Liberty}® cycler. Once the connectors are connected, the patients, as they currently do, communicate to the machine that they have connected all pertinent lines, and are ready to proceed. The Liberty® cycler then goes through the automated process of attaching the disposable guide wire 130 to the guide wire catch 190. The process for this is as follows:

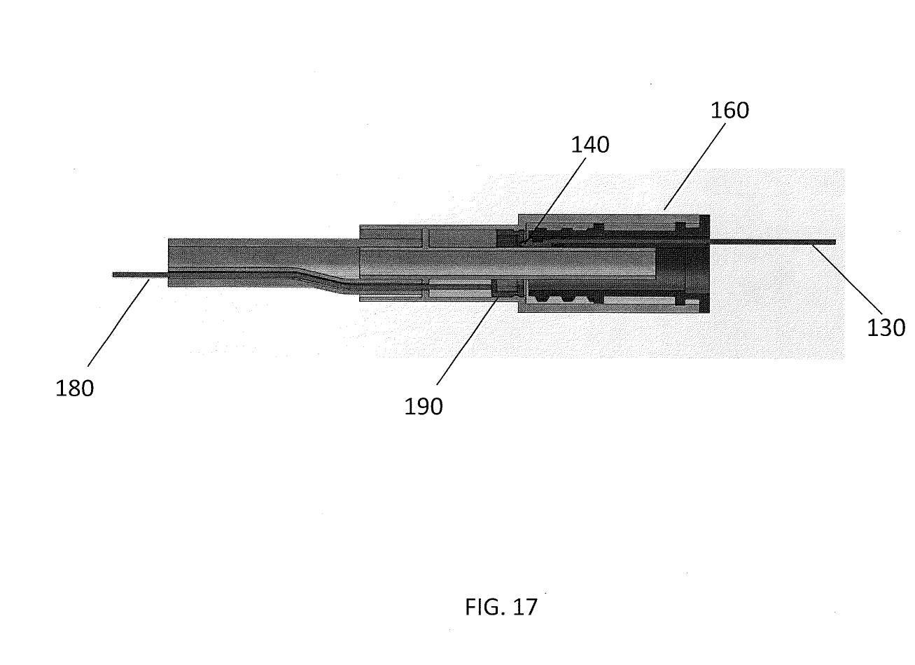

1. The machine pushes the disposable guide wire 130 out and into the guide wire catch 190 as shown in FIG. 17.

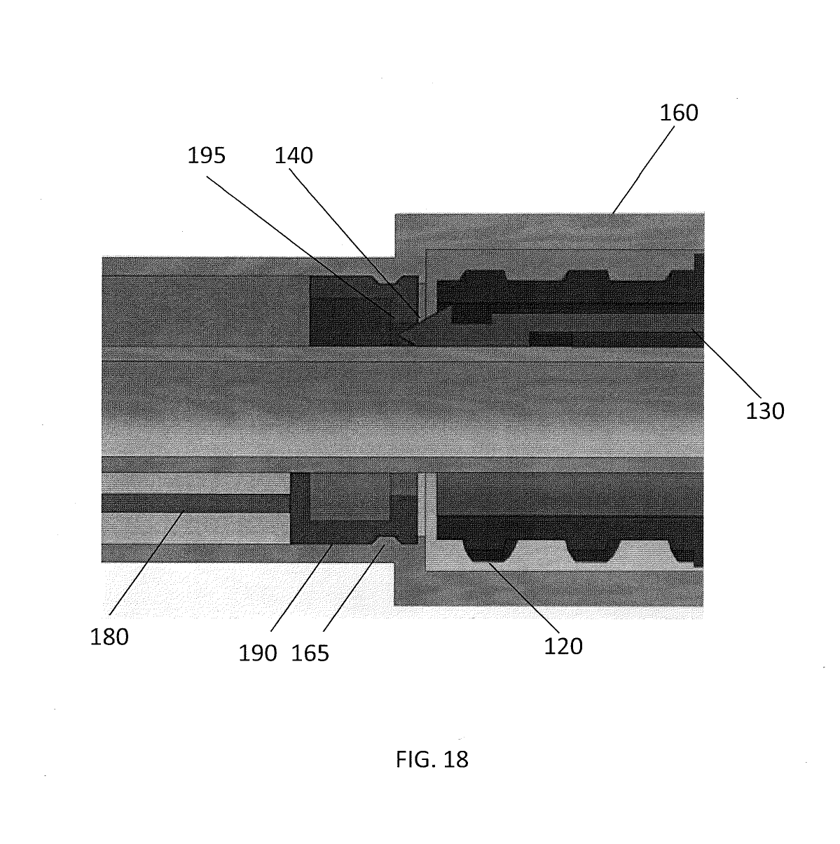

2. The guide wire catch 190 is locked at this point, as shown in FIG. 18. The lock 165 is a stepped feature which when locked sits in a pocket (i.e., groove 175 shown in FIG. 14) in the guide wire catch 190.

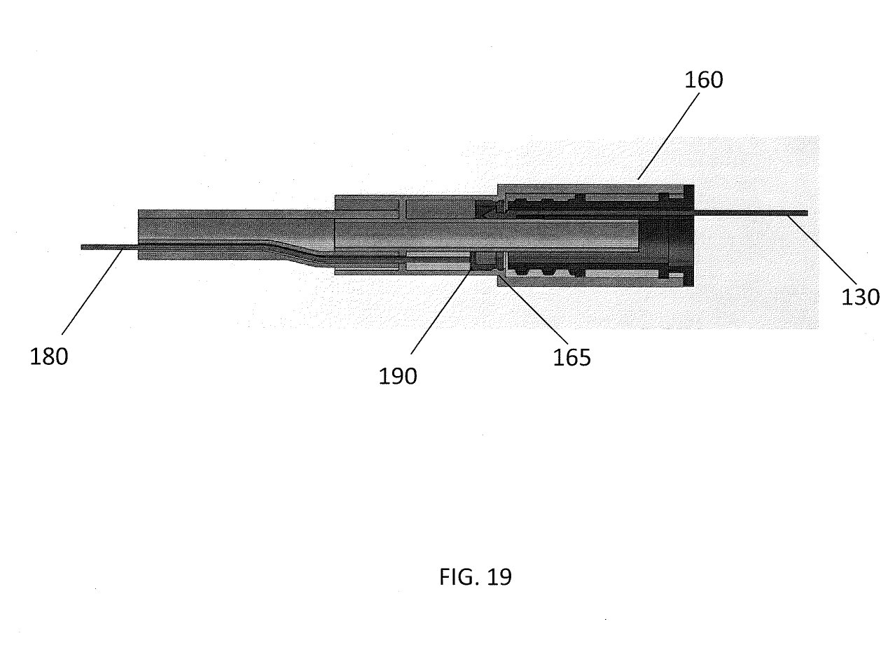

3. The machine continues to push the disposable guide wire 130 until the wire barb 140 snaps past the lip 195 of the guide wire catch 190. The guide wire catch lock 165 will hold the guide wire catch 190 in place with enough force to withstand the attachment of the disposable guide wire 130. The disposable guide wire 130 and the guide wire catch 190 are now connected, as shown in FIG. 19.

4. Once connected, the machine will exert enough force on the disposable guide wire 130 to push the guide wire catch 190 past the guide wire catch lock 165.

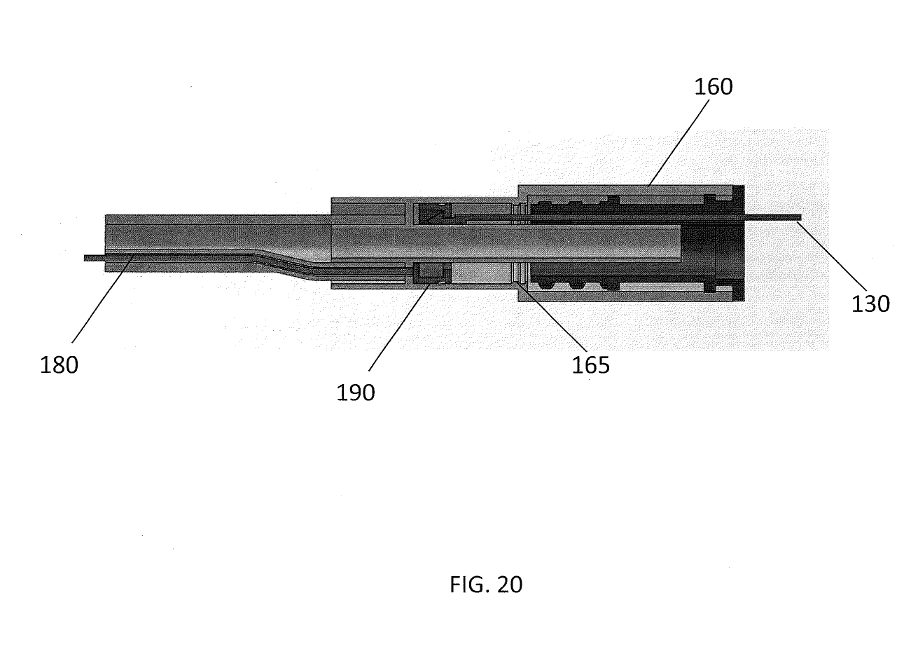

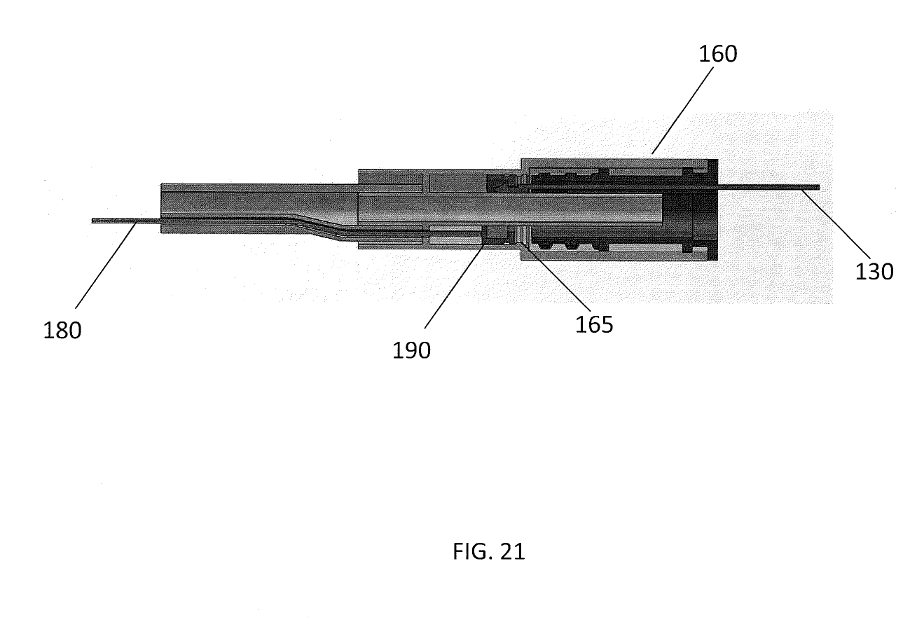

5. Once past the lock 165, the machine can move the disposable guide wire 130 forward and backward and in turn move the patient guide wire 180 forward and backward, as shown in FIGS. 20 (forward) and 21 (backward). As shown in FIG. 21, the normal motion of the guide wire catch 190 is outside of the locking feature 165. As a result the locking feature 165 does not interfere with the normal motion of the mechanism.

The basic components of the catheter assembly 100 for the double wire version are the same, including two each of disposable guide wires 130a and 130b, disposable guide wire barbs 140a and 140b, guide wire catches 190a and 190b, and patient guide wires 180a and 180b.

The attachment method between the patient and the disposable is very simple. To the patients, there will be no extra operation required beyond the simple screw connection they already make. All of the connection components are housed within the patient catheter 150 and the disposable connector 110.

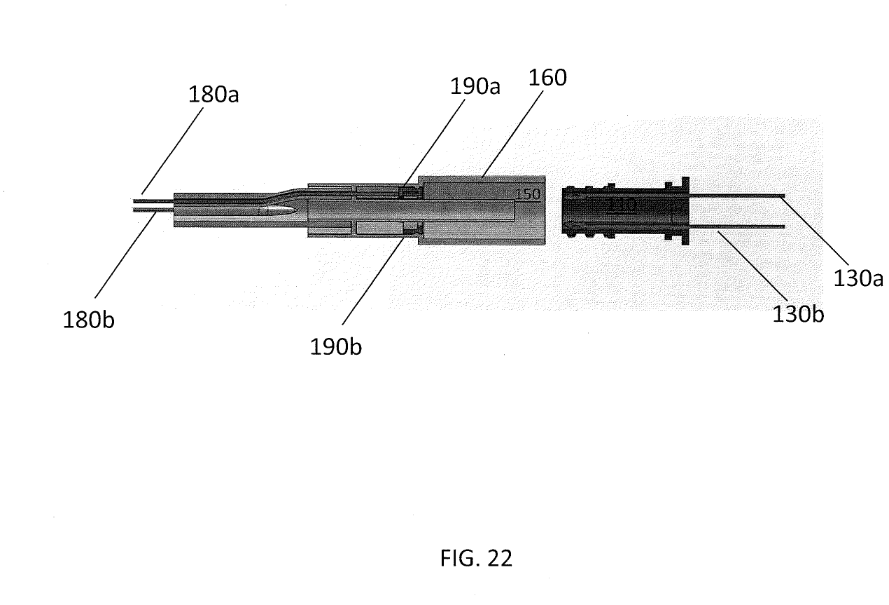

FIG. 22 shows the assembly as it would exist before connection of the patient connector 160 to the disposable connector 110. The patients, during connection of the disposable connector 110 to the patient connector 160, would connect as they currently do with the simple screw connectors. No extra action is required.

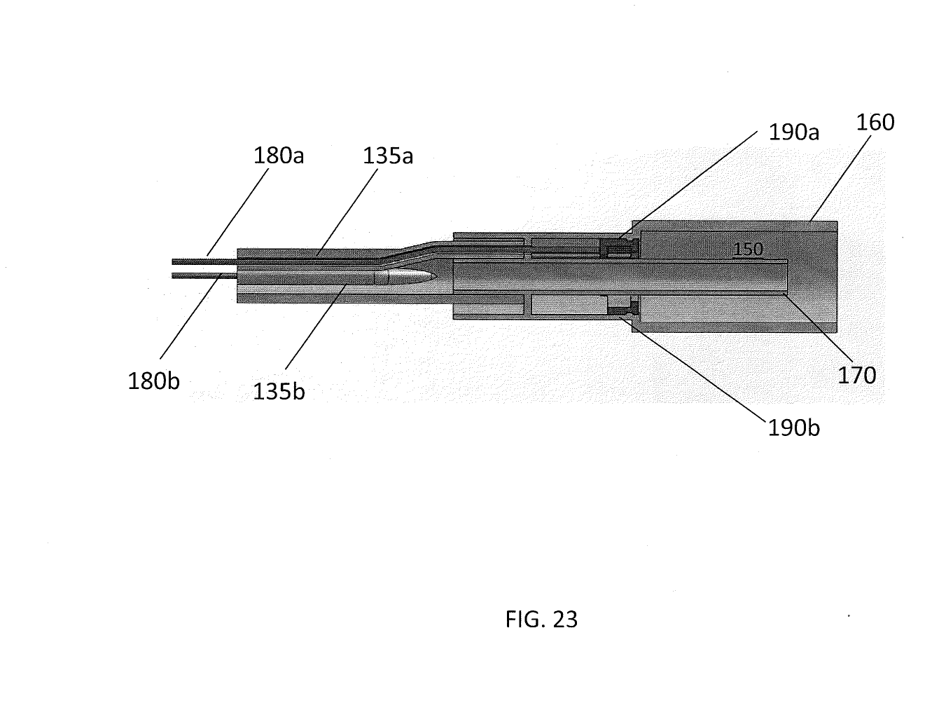

The disposable connector 110 has a similar construction to the existing connector currently in use with the Liberty® cycler cassette. A close up illustration of the disposable connector 110 including two external guide wires 130 and 130b is shown in FIG. 1B, including the two internal channels 125a and 125b for the guide wires 130a and 130b to ride within. The channels 125a and 125b stop short of the end of the connector 110, allowing room for the barbs 140a and 140b on the end of the disposable guide wires (i.e., barbs 140a and 140b used for connecting the disposable guide wires 130a and 130b to the guide wire catches 190a and 190b). When disconnected, the disposable guide wires and disposable barbs are fully within the disposable connector, preventing injury to the patient from the barbs, and preventing damage to the barbs. The two-wire patient catheter 150 houses the active parts enabling the connection between the disposable guide wires 130a and 130b and the patient guide wires 180a and 180b. A close up illustration of the two-wire patient catheter 150 is shown in FIG. 23.

As shown in FIG. 23, the patient catheter 150 houses the guide wire catches 190a and 190b, which connect the patient guide wires 180a and 180b to the disposable guide wires 130a and 130b. The patient catheter 150 has channels 135a and 135b within it for the patient guide wires 180a and 180b to ride within. The patient guide wires 180a and 180b are connected to the guide wire catches 190a and 190b, pass through small holes in the wall of the patient catheter 150, separating the connection area from the tubing connection area, and into the rest of patient catheter 150. Any motion of the guide wire catches 190a and 190b is transmitted to the patient guide wires 180a and 180b.

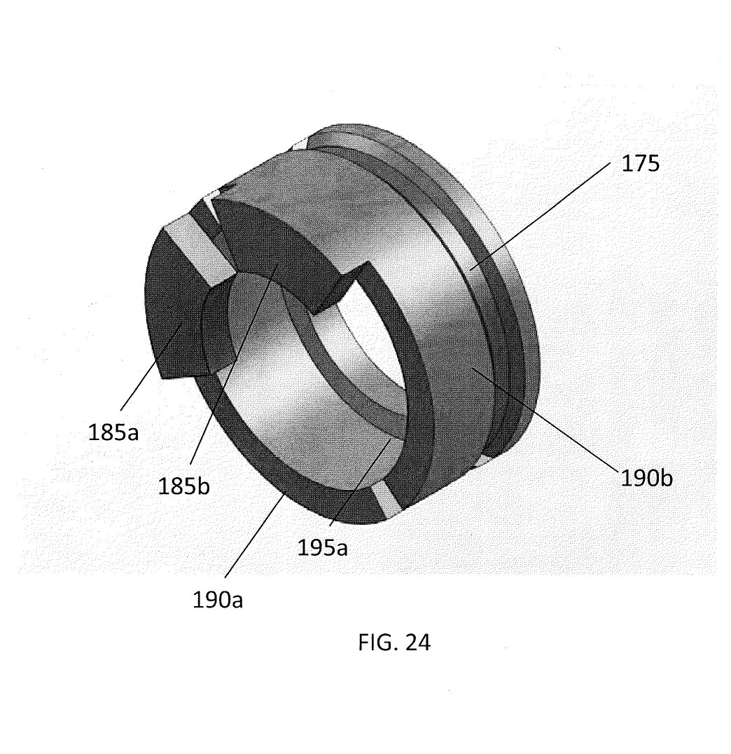

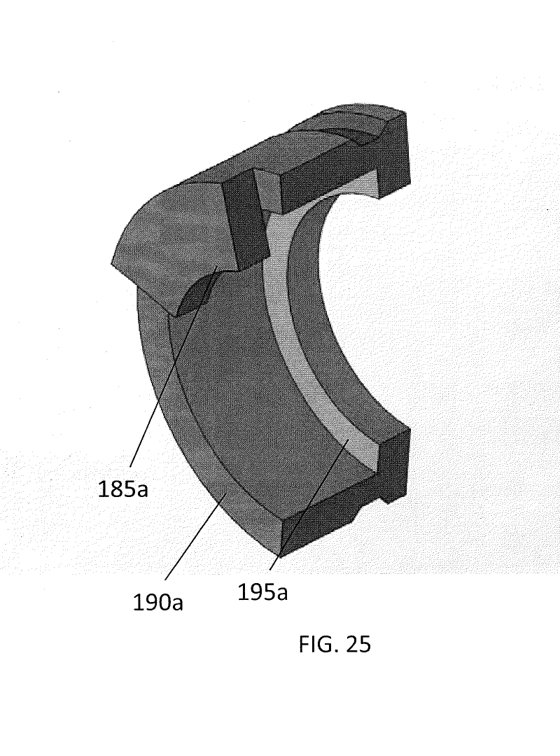

The purpose of the guide wire catches 190a and 190b is to transmit the pushing and pulling forces from the disposable guide wires (i.e., external guide wires) 130a and 130b to the patient guide wires (i.e., internal guide wires) 180a and 180b. They also have to allow for the disposable guide wires 130a and 130b to connect and disconnect easily, without patient interaction, ideally not altering the catheter/disposable connection method currently used by patients. The current method of catheter/disposable connection is by way of a simple screw fitting. As a result, the guide wire catches must allow for the disposable guide wires to connect in any position in a 360 degree circle around the axis of the connectors (alignment is not guaranteed since the patient is screwing one fitting into another, and the fittings are double started). One result of these restrictions is the guide wire catches 190a and 190b, shown close up in FIGS. 24 and 25.

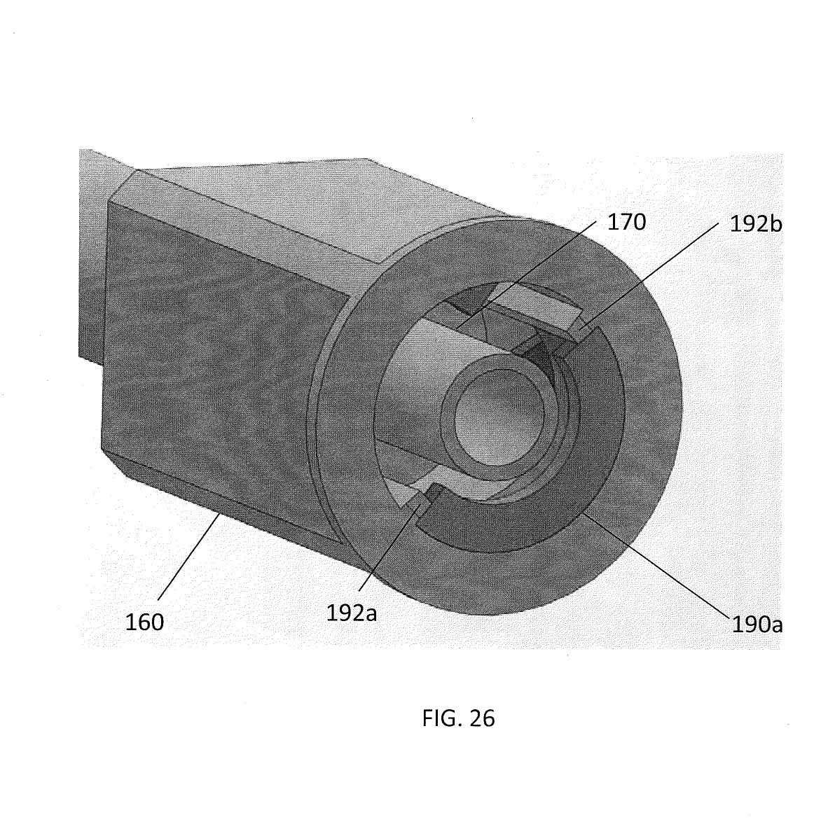

In the double wire version, there are two guide wire catches 190a and 190b. FIG. 25 shows both catches 190a and 190b as they would be oriented in the patient connector 150. The guide wire catches are sized such that only one disposable guide wire 130a or 130b (not shown) can connect to a guide wire catch at a time. The disposable guide wires 130a and 130b are about 180 degrees offset from one another, as shown in FIG. 1B, while the guide wire catches 190a and 190b only cover about 170 degrees, as shown in FIG. 26, with the remaining about 10 degrees being taken up by guide rails 192a and 192b described further below. Each guide wire catch operates independently, allowing for fully independent motion of the patient guide wires.

As shown in FIG. 24, the guide wire catch 190a has a small tab 185a on the back to allow connection to the patient guide wire 180a (not shown). The small lips 195a (and 195b (not shown)) seen in FIGS. 24 and 25 are catches to hold the disposable guide wire barbs 140a and 140b, and allow for the forward and backward movement of the guide wire catches 190a and 190b, and as a result, the patient guide wires 180a and 180b.

As shown in FIG. 24, the guide wire catches 190a and 190b also have the same locking groove 175 as was present in the single wire version. The operation of the groove is identical to the single wire version.

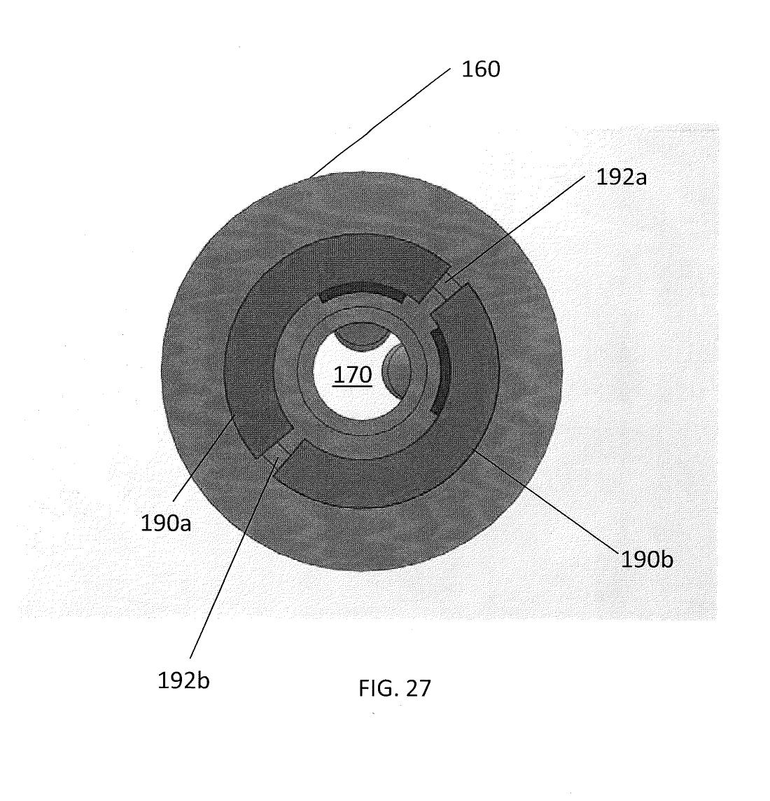

As shown in FIGS. 26 and 27, there are gaps between the catches (only 190a is shown in FIG. 26 for clarity) for guide rails 192a and 192b molded into the patient connector 160 to fit between the catches 190a and 190b. The guide rails keep the guide wire catches from rotating within the patient connector.

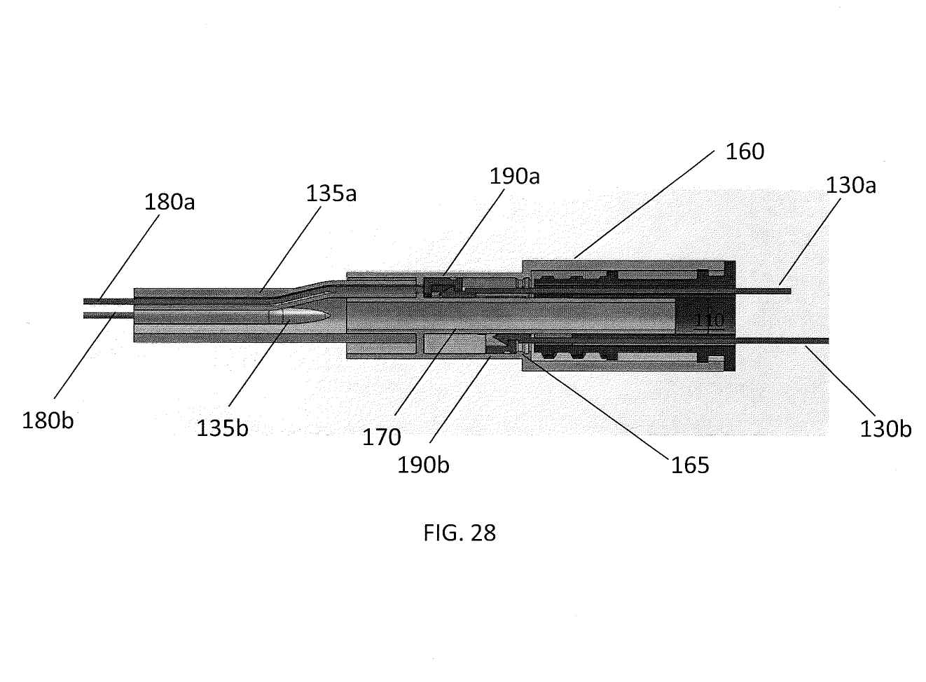

The guide wire catch anti-rotation guide rails 192a and 192b prevent the guide wire catches 190a and 190b from rotating within the patient connector 160, keeping all movement of the guide wire catches linear. Connection of the disposable connector and the patient connector is the same as described above for the single wire connection. The guide wire catches 190a and 190b are moved independently by actuation of the disposable guide wires 130a and 130b. FIG. 28 shows the two-wire assembly with guide wire catch 190a all the way in and guide wire catch 190b all the way out (but free of guide wire catch lock 165). In the same manner as the single wire version, the normal motion of the guide wire catch is outside of the locking feature. As a result the locking feature does not interfere with the normal motion of the mechanism.

In either the single wire or the two wire version, the Liberty® cycler will automatically disconnect the disposable guide wires 130a and 130b from the guide wire catches 190a and 190b. The process of disconnecting is as follows:

1. The cycler will begin to return the active catheter to the locked position at a time determined by the travel of the active catheter during treatment. If the catheter had a long travel during treatment, the cycler will start the return travel earlier, while if the travel was shorter, the cycler will start the return travel later (closer to the end of the treatment).

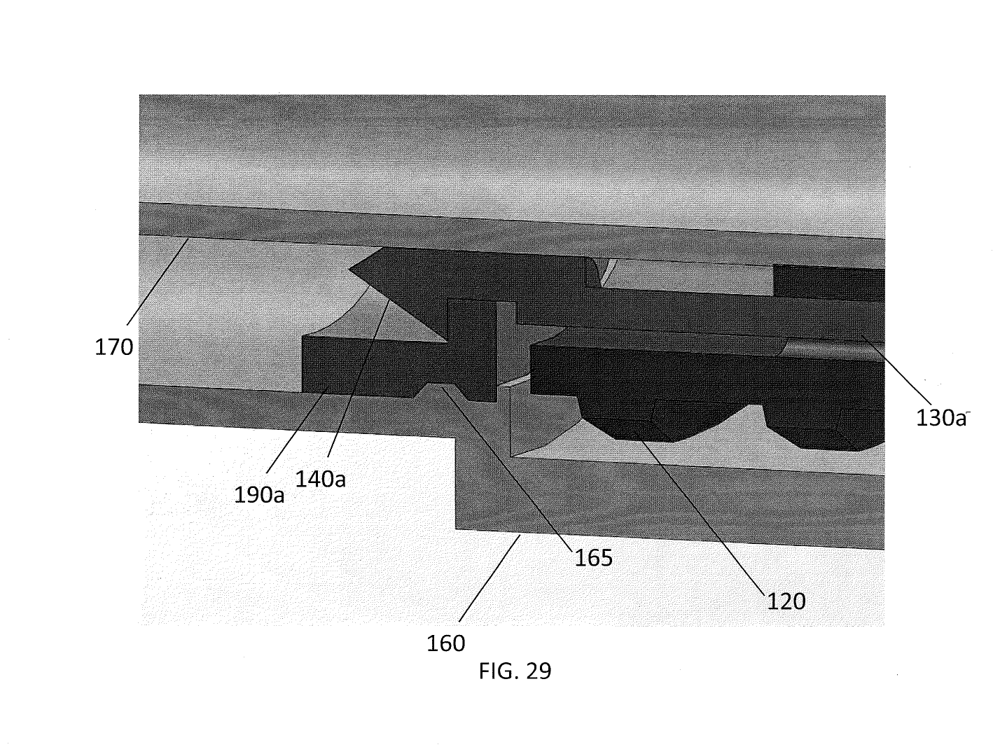

2. The cycler will pull the guide wire catches 190a and 190b up over the locking feature 165, locking the guide wire catches in place, as shown in FIG. 29.

The Liberty® cycler retracts the disposable guide wires until the barbs snap free of the guide wire catches (similar to how a snap button on a jacket works). The Liberty® Cycler continues to retract the disposable guide wires until they are fully retracted within the disposable connector. Once the disposable guide wires are fully retracted, the Liberty® cycler notifies the patient that they are now able to finish their treatment by disconnecting themselves from the disposable connector.

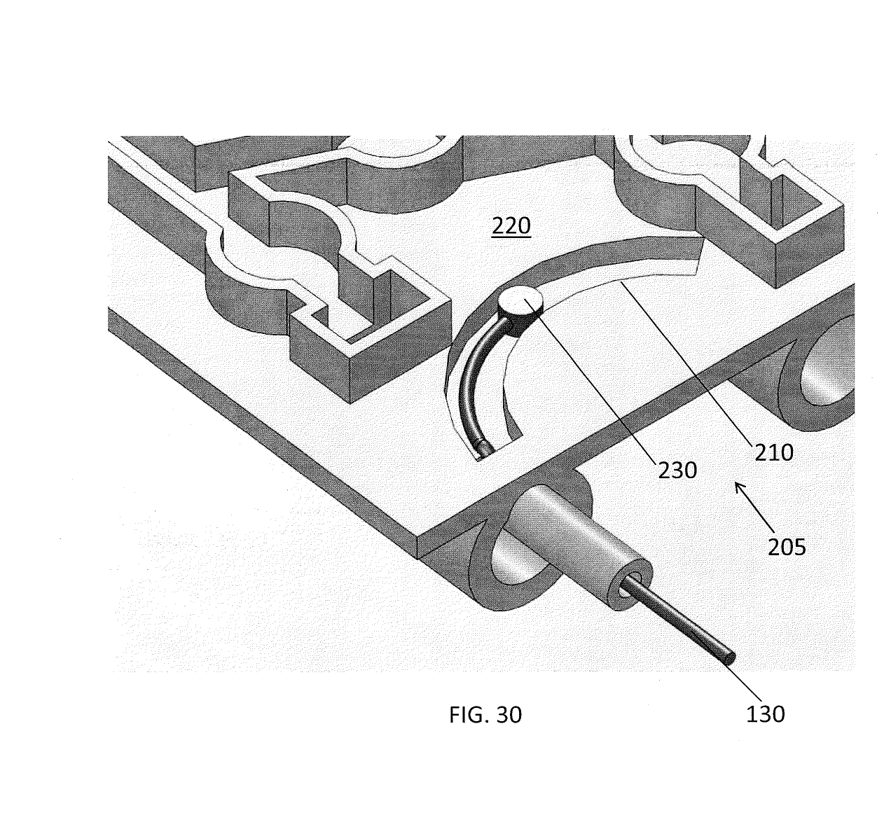

As shown in FIGS. 10 and 30, the basic components of the active catheter cassette are as follows (with examples of suitable respective materials):

1. Liberty® Cycler cassette 220 with disposable guide wire movement pocket 210 (polypropylene)

2. disposable guide wire 130 (other end of wire from disposable connector assembly) (stainless steel or plastic filament)

3. disposable guide wire magnet sink 230 (magnetic stainless steel or coated plain steel)

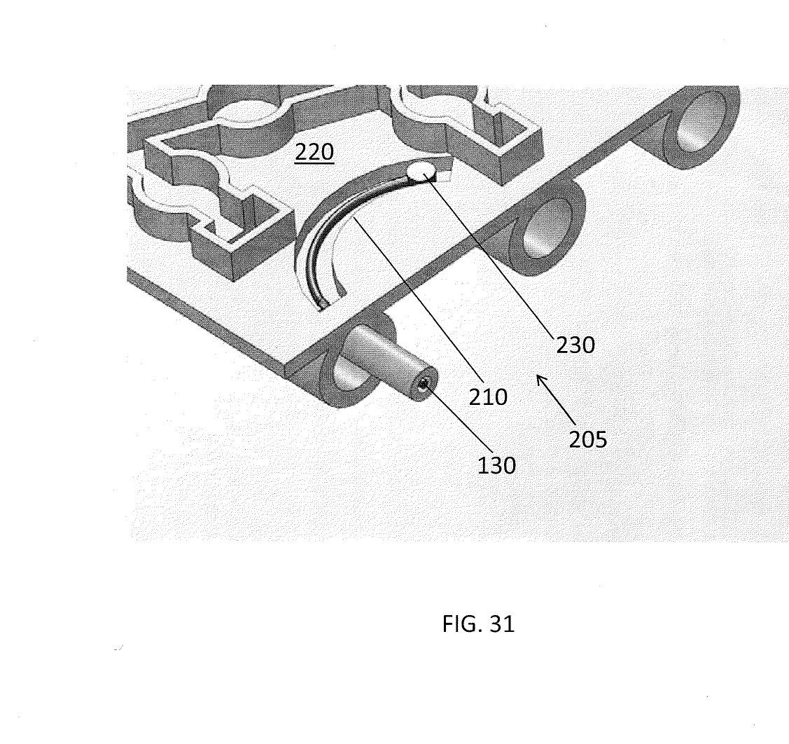

The Liberty® Cycler moves the disposable guide wire by means of a magnet and a special pocket molded into the cassette. The assembly is shown in FIGS. 10 and 30. The guide wire actuator assembly 205 has a small pocket 210 molded in to contain the cassette end of the disposable guide wire 130 and the disposable guide wire magnet sink 230. The magnet sink 230 is attached to the end of the disposable guide wire 130. The magnet sink 230 serves as a large ferrous mass for a magnet (such as a single electromagnet, an array of electromagnets, a single permanent magnet, a magnetic field producing induction coil, etc.), embedded in the pump body (not shown), to attract to the magnet sink 230. As the magnet embedded within the machine moves (or changes the magnetic field) it induces a movement from the magnet sink 230, forcing it to move along its path. By forcing the magnet sink 230 to move (such as in this case in a small arc 210), the disposable guide wire 130 is forced to move forward and back as shown in FIGS. 31 (wire 130 retracted) and 32 (wire 130 extended). As the magnet sink 230 moves back and forth, the disposable guide wire 130 retracts and extends. The use of a magnetic connection allows there to be no physical connection between the disposable guide wire and its motion source. In this embodiment, the disposable guide wire/magnet sink assembly can be covered with a thin polypropylene film. The assembly could also be covered with a thin hard plastic window.

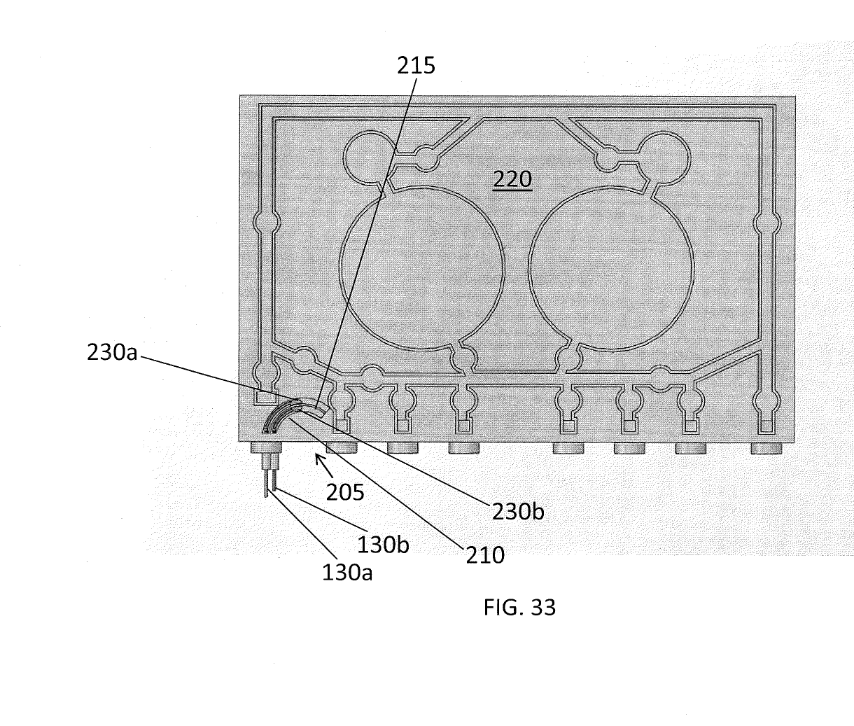

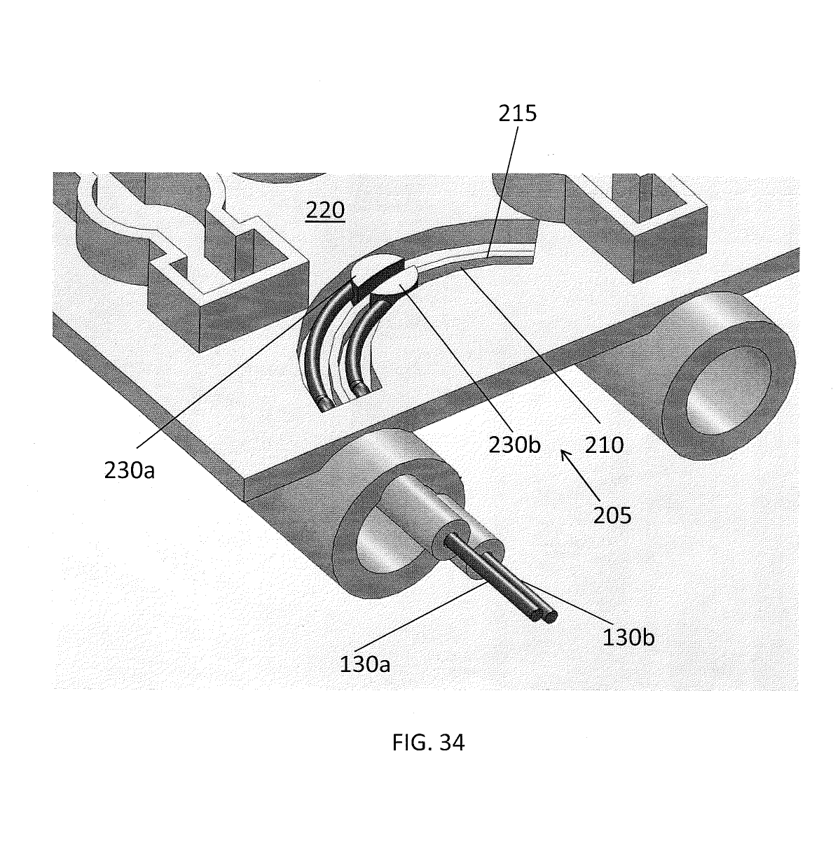

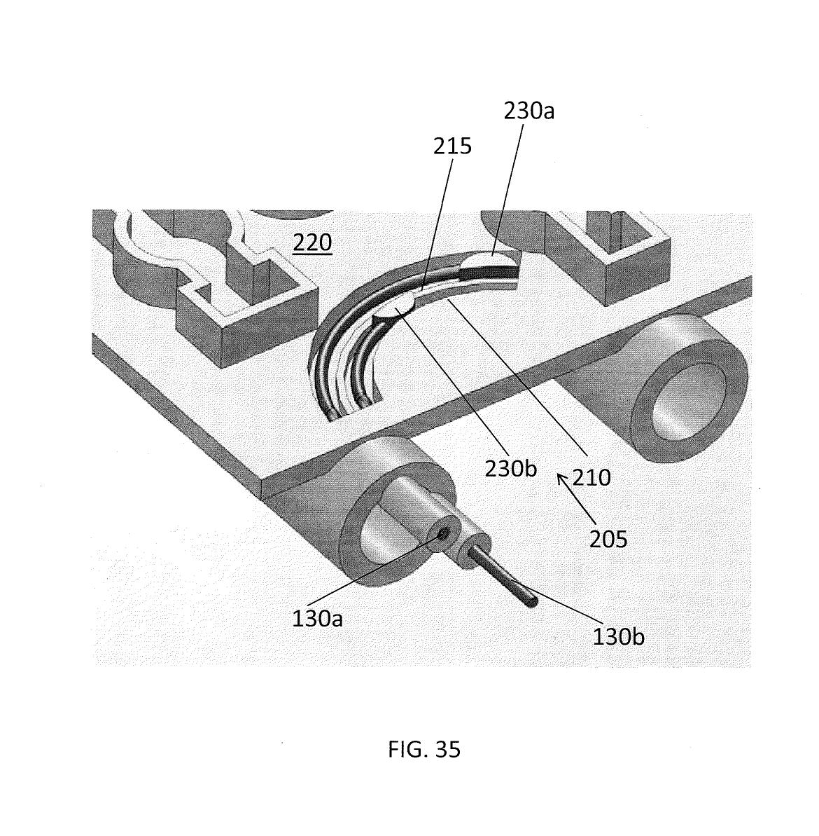

The basic components of the active catheter cassette for the double wire version are the same as for the single-wire version, except for a modification of the movement pocket 210 in the cassette to accommodate the two disposable wires 130a and 130b and corresponding two magnet sinks 230a and 230b, as shown in FIGS. 33 and 34. In the two-wire version, as shown in FIG. 35, the magnet sinks 230a and 230b are independent, and as a result are able to be actuated independently. The magnet pocket divider 215 separates the magnet sinks.

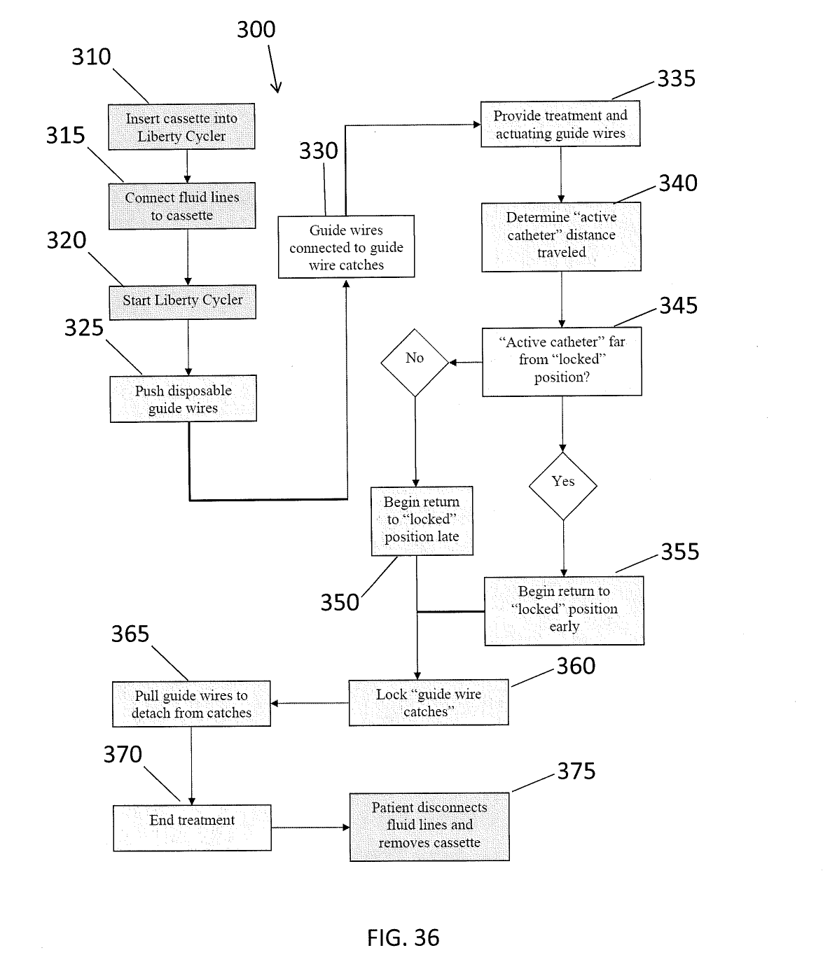

As shown in FIG. 36, flow chart 300 illustrates a method of articulating a catheter that includes, at step 310, inserting a cassette into a Liberty cycler, and, at step 315, connecting liquid lines to the cassette by inserting a tubular member having a first connector at its distal end and at least one external guide wire that is slidably contained inside the tubular member into a proximal end of a catheter, the catheter housing a liquid-flow tube. At step 320, the method includes starting the Liberty cycler, and, at step 325, extending the at least one external guide wire until the at least one guide-wire catch engages the external guide wire at step 330, thereby forming a guide-wire assembly. At step 335, the method includes providing peritoneal dialysis treatment to a patient and actuating the guide wires by moving the guide-wire assembly longitudinally to articulate the catheter. Navigation of the catheter can be accomplished by passive computer algorithm, or, alternatively, by active imaging using ultrasound or other similar imaging technology. The method then includes, at step 345, a determination of whether the active catheter has moved far from the locked position. If the catheter had a long travel during treatment, then, at step 355, the cycler begins returning to the locked position early during the treatment, otherwise, at step 350, the cycler begins returning to the locked position at a time closer to the end of the treatment. At step 360 the cycler locks the guide wire catches, and, at step 365, the cycler pulls the guide wires to detach the guide wires from the guide wire catches. At step 370, the cycler ends the treatment, and, at step 375, the patient disconnects the liquid lines and removes the cassette.

The relevant teachings of all patents, published applications and references cited herein are incorporated by reference in their entirety.

While this invention has been particularly shown and described with references to example embodiments thereof, it will be understood by those skilled in the art that various changes in form and details may be made therein without departing from the scope of the invention encompassed by the appended claims.

What is claimed is:

1. A catheter assembly comprising a tubular member having a first connector at its distal end for coupling with the proximal end of a catheter, the tubular member having at least one external guide wire slidably contained inside the tubular member, the at least one external guide wire having an attachment member for attaching to an internal guide wire catch slidably contained inside the catheter, whereby manipulation of the guide wire causes motion in the distal end of the catheter.

2. The catheter assembly of claim 1, wherein the first connector is a male connector, and the proximal end of the catheter comprises a female connector.

3. The catheter assembly of claim 1, further including a catheter for flowing liquid into and out of an internal cavity of a patient, the catheter housing a liquid-flow tube, at least one internal guide wire slidably contained inside the liquid-flow tube, and at least one guide-wire catch slidably contained inside the catheter.

4. The catheter assembly of claim 3, wherein the liquid is dialysis solution, and the internal cavity is the peritoneal cavity of the patient.

5. The catheter assembly of claim 3, wherein the at least one guide-wire catch includes a tab configured to connect to the internal guide wire.

6. The catheter assembly of claim 3, wherein the attachment member on the external guide wire is angled to slidably connect to a step on the guide wire catch.

7. The catheter assembly of claim 3, further including at least one wire guide located within, positioned along, and connected to an inside surface of the liquid-flow tube, wherein the at least one internal guide wire is slidably contained within the at least one wire guide, and is connected to the liquid-flow tube at a distal end of the wire guide.

8. The catheter assembly of claim 7, wherein the wire guide is a tube, the tube being closed at the distal end.

9. The catheter assembly of claim 7, wherein the wire guide is a series of tabs with guide openings.

10. The catheter assembly of claim 7, wherein the at least one wire guide includes two wire guides positioned at 90 degrees from each other with respect to the center of the liquid-flow tube, each wire guide including one internal guide wire slidably contained within the wire guide, and connected to the liquid-flow tube at the distal end of the wire guide.

11. A catheter system comprising:
a tubular member having a first connector at its distal end for coupling with a proximal end of a catheter, the tubular member having at least one external guide wire slidably contained inside the tubular member, the at least one external guide wire having an attachment member for attaching to an internal guide wire catch slidably contained inside the catheter;
a catheter housing a liquid-flow tube, at least one internal guide wire slidably contained inside the liquid-flow tube, and at least one guide-wire catch slidably contained inside the catheter, whereby manipulation of the guide wire causes motion in the distal end of the catheter; and
a guide wire actuator.

12. The catheter system of claim 11, wherein the first connector is a male connector, and the proximal end of the catheter comprises a female connector.

13. The catheter system of claim 11, wherein the at least one guide-wire catch includes a tab configured to connect to the internal guide wire.

14. The catheter system of claim 11, wherein the attachment member on the external guide wire is angled to slidably connect to a step on the guide wire catch.

15. The catheter system of claim 11, further including at least one wire guide located within, positioned along, and connected to an inside surface of the liquid-flow tube, wherein the at least one internal guide wire is slidably contained within the at least one wire guide, and is connected to the liquid-flow tube at a distal end of the wire guide.

16. The catheter assembly of claim 15, wherein the wire guide is a tube, the tube being closed at the distal end.

17. The catheter assembly of claim 15, wherein the wire guide is a series of tabs with guide openings.

18. The catheter system of claim 15, wherein the at least one wire guide includes two wire guides positioned at about 90 degrees from each other with respect to the center of the liquid-flow tube, each wire guide including one internal guide wire slidably contained within the wire guide, and connected to the liquid-flow tube at the distal end of the wire guide.

19. The catheter system of claim 11, wherein the guide wire actuator includes an apparatus for pumping dialysis solution between a peritoneal dialysis machine and a patient, in order to perform peritoneal dialysis on the patient.

20. A method of articulating a catheter comprising:
inserting a tubular member having a first connector at its distal end and at least one external guide wire slidably contained inside the tubular member into a proximal end of a catheter, the catheter housing a liquid-flow tube, at least one internal guide wire slidably contained inside the liquid-flow tube, and at least one guide-wire catch slidably contained inside the catheter, thereby forming a catheter assembly;
extending the at least one external guide wire until the at least one guide-wire catch engages the external guide wire, thereby forming a guide-wire assembly; and
moving the guide-wire assembly longitudinally to articulate the catheter.

21. The method of claim 20, wherein the catheter assembly includes a second guide-wire assembly.
